(19) United States
(12) Patent Application Publication
(10) Pub. No.: US 2025/0263518 A1
(43) Pub. Date: Aug. 21, 2025

(54) FUNCTIONALIZED POLYMERIC MATERIAL FOR FORMING NITRIC OXIDE AND METHODS THEREFOR

(71) Applicant: Sterile State, LLC, Farmington Hills, MI (US)

(72) Inventor: Megan Cecelia Frost, Hancock, MI (US)

(73) Assignee: Sterile State, LLC, Farmington Hills, MI (US)

(21) Appl. No.: 18/857,169

(22) PCT Filed: Apr. 18, 2023
(86) PCT No.: PCT/US2023/018910

§ 371 Date: Oct. 15, 2024

Related U.S. Application Data
(60) Provisional application No. 63/332,871, filed on Apr. 20, 2022.

(51) Int. Cl. C08G 18/87; A01N 59/00; A01P 1/00; B01J 31/12; C08F 218/08; C08G 18/44; C08G 77/392; C08G 77/458

(52) U.S. Cl. CPC C08G 18/87; A01N 59/00; A01P 1/00; B01J 31/12; C08F 218/08; C08G 18/44; C08G 77/392; C08G 77/458

(57) ABSTRACT

A modified polymeric material is presented that contains substantial quantities of nitric oxide precursor groups within and on the surface of a variety of polymeric materials. Advantageously, the nitric oxide precursor groups are covalently bound to the oxygen atoms of carbonyl or siloxyl groups in the polymer via a functionalized organosilane, thereby enabling large quantities in the polymer. In further desirable aspects, the modifications can be introduced into already produced polymeric materials using a conceptually simple and simple sequence of reactions.

### CROSS REFERENCE TO RELATED APPLICATIONS

This application claims priority to our copending US provisional patent application with the Ser. No. 63/332,871, which was filed Apr. 20, 2022, and which is incorporated by reference herein.

### FIELD OF THE INVENTION

The present disclosure relates to functionalized polymeric material comprising pendant reactive groups that are modified to form pendant nitric oxide precursor groups from which nitric oxide can be released in antimicrobially effective quantities.

### BACKGROUND

A variety of products and articles, including, for example, medical instruments, devices, and equipment, must be sterilized prior to use to prevent microbial contamination of a wound site, or a biological sample, etc. To that end, a number of sterilization processes are known that in many cases involve a step of contacting the product or article with a sterilant. Examples of such sterilants include dinitrogen tetroxide, steam, ethylene oxide, hydrogen peroxide, dry heat, and the like.

Unfortunately, some of the known sterilants will chemically adversely react with a number of materials and are therefore not suitable for use, while other sterilants can be applied but will then relatively quickly react or otherwise decompose. To overcome such difficulties, antimicrobial agents such as zinc complexes, colloidal silver, or triclosan can be included into a material to so provide at least some antimicrobial effect. However, such antimicrobial agents may be problematic for use with medical devices or articles that contact food.

In further examples of sterilization of an article, gaseous nitric oxide may be formed in an enclosure from a precursor such as a nitrosothiol to so expose the article to sterilizing quantities of nitric oxide as is described in WO 2022/164894, using materials prepared as described in U.S. Pat. No. 9,884,943. Unfortunately, due to the coupling chemistry for the nitric oxide precursor groups used in the '894 and '943 references, the quantity of nitric oxide precursor groups in the polymer is limited. Moreover, the nitric oxide precursor groups in these references must be introduced into the polymer at the time of polymerization, which further restricts their usefulness. Similar difficulties arise with modified supramolecular polymer complexes that are modified to include a nitrosothiol as the nitric oxide precursor groups as disclosed in US 2010/0303891. In yet another related example, surface modified nitric oxide releasing nanoparticles of US 2011/0151000 rely on terminal hydroxyl groups for the covalent attachment of the nitric oxide precursor group. As such, these modified polymers may be suitable for nitric oxide release in vivo to effect nitric oxide modulated processes (e.g., regulation of vascular tone, platelet aggregation, etc.), however, will generally be unsuitable for microbial control, particularly over extended periods.

Accordingly, there remains an opportunity for improved polymeric materials capable of releasing nitric oxide from a precursor group in antimicrobially effective quantities for a variety of medical and consumer purposes, and particularly for manufacture of such materials where the polymer is already produced and subsequently modified to include pendant nitric oxide precursor groups.

### BRIEF SUMMARY OF THE INVENTION

The inventor has discovered functionalized polymeric materials exhibiting improved properties with respect to quantities of nitric oxide that can be released and simplified manufacture of such polymeric materials. More particularly, the compounds, compositions, and methods presented herein allow for functionalization of already manufactured polymers (e.g., medical or food grade polymers) in a manner that allows introduction of nitric oxide precursor groups that can release nitric oxide in antimicrobially effective amounts over extended periods of time.

In preferred aspects, an already manufactured polymer includes a plurality of carbonyl groups in which the oxygen of respective carbonyl groups participates in a catalytic reaction with an aminosilane to so introduce a pendant amino group into the polymer. The so introduced amino group is then reacted with and covalently bound to a reagent that contains a thiol group, which is subsequently reacted with a nitrite or nitric oxide in gas phase to so form the corresponding S-nitrosothiol as the pendant nitric oxide precursor group. Alternatively, or additionally, the oxygen of respective carbonyl groups in the polymer participates in a catalytic reaction with an epoxy silane to so introduce a pendant epoxy group into the polymer. The so introduced epoxy group is then reacted with and covalently bound to a reagent that contains a thiol group, which is subsequently reacted with a nitrite to so form the corresponding S-nitrosothiol as the pendant nitric oxide precursor group.

As will be readily appreciated, suitable carbonyl groups may be located in an amide bond of a polyurethane, in a carbonate group of a polycarbonate, or in a pendant acetate group of an ethylene-vinyl acetate (EVA) polymer. In addition, contemplated catalytic reactions may also be performed in polymers that include siloxyl (but not silanol) groups, which may be located in various substituted or unsubstituted polysiloxanes. Therefore, and viewed from a different perspective, especially contemplated (already manufactured) polymers will include polyurethanes, polyamides, EVA, polycarbonates, polysiloxanes (e.g., polydimethylsiloxane) etc., and all co(block)polymers containing such polymers.

Therefore, in some aspects of the inventive subject matter, functionalized polymeric materials may comprise a pendant amino- or epoxy-functional group that may subsequently be used as an anchor for a pendant nitric oxide precursor group, which is capable of decomposing to form nitric oxide. The inventor contemplates that the functionalized polymeric material with the pendant nitric oxide precursor group may be utilized as, or to form, an object that is cast, molded, or milled, a film, a coating, or combinations thereof. In various embodiments, the functionalized polymeric material with the pendant nitric oxide precursor group therefore allows for safe, efficient, and environmentally friendly gas sanitation, germ reduction, or even sterilization of a wide range of articles such as medical devices, medical equipment, endoscopes, cell phones, mask coverings, keys, name badges, credit cards, mouth guards, baby pacifiers and teething rings, pens and pencils, sports gloves, shoes, etc. The functionalized polymeric material with the pendant nitric oxide precursor group will advantageously release gas phase nitric oxide under ambient temperature, pressure, and humidity, which may be further enhanced by illumination with visible and UV light as well as by an increase in temperature.

In some embodiments, the inventor contemplates that the functionalized polymeric material comprises the reaction product of a base polymeric material and an aminosilane or epoxy silane, most preferably by (organometal catalyzed) reaction of the aminosilane and/or epoxy silane with a carbonyl oxygen present in the base polymeric material. The base polymeric material and the aminosilane thus react to introduce a pendant amino- and/or epoxy-functional group into the base polymeric material. In at least some cases, the so functionalized polymeric material may exhibit an increased solubility in organic solvent (e.g., ethyl acetate, propyl acetate, hexanes, etc.) as compared to the solubility of the base polymeric material as determined in accordance with ASTM 3132-84.

In other embodiments, the functionalized polymeric material may comprise the reaction product of the base polymeric material, the amino- or epoxy silane, and a nitric oxide precursor. The base polymeric material, the silane, and the nitric oxide precursor react to form the pendant nitric oxide precursor group on the base polymeric material. Notably, in various embodiments, the pendant nitric oxide precursor group exhibits an improved stability as compared to the stability of the nitric oxide precursor separate from the polymer. In these and other embodiments, the pendant nitric oxide precursor group exhibits a reduced rate of decomposition to nitric oxide as compared to rate of decomposition of the nitric oxide precursor to nitric oxide without being bound to the base polymeric material.

The inventor contemplates that the base polymeric material has a carbonyl-functional group, a siloxy-functional group, or combinations thereof. To this end, the base polymeric material may include a moiety/repeat unit that comprises the carbonyl-functional group or the siloxy-functional group. For example, the moiety may be a urethane moiety, an ester moiety, a carbonate moiety, a siloxane moiety, or combinations thereof. In still further aspects, the base polymeric material is substantially free of hydroxyl-functional groups.

In additional embodiments, the moiety comprises the carbonate moiety and the siloxane moiety in various arrangements (e.g., a polycarbonate-polydimethylsiloxane block copolymer). In yet other embodiments, the moiety/repeat unit comprises the urethane moiety and the siloxane moiety in various arrangements (e.g., a polyurethane-polydimethylsiloxane block copolymer). In still other embodiments, the moiety/repeat unit comprises only a urethane moiety (e.g., polyurethane), or only a siloxane moiety (e.g., a polydimethylsiloxane), or only an ester moiety (e.g., a poly(ethylene-co-vinyl acetate) copolymer).

The inventor further contemplates that the silane has an amino-functional group or an epoxy-functional group. In various embodiments, the silane comprises an aminoalkoxysilane (e.g., a 3-aminopropyl trimethoxy silane). It is contemplated herein that the reaction product of the base polymeric material and the silane may be formed in the presence of a catalyst. The catalyst may comprise a transition metal catalyst (e.g., an organotin catalyst). It is further contemplated herein that the reaction product of the base polymeric material and the silane may be formed in the presence of a solvent. The solvent may comprise an organic solvent (e.g., tetrahydrofuran). It is to be appreciated that the reaction product of the base polymeric material, the silane, and the nitric oxide precursor may be formed in the presence of the catalyst, the solvent, or a combination thereof.

In some embodiments, the nitric oxide precursor comprises the reaction product of an acetylpenicillamine thiolactone and a nitrosating compound, such as a nitrite. The nitrite may comprise sodium nitrite, calcium nitrite, potassium nitrite, tetrabutylammonium nitrite, dicyclohexylammonium nitrite, butylnitrite, isobutylnitrite, t-butylnitrite, amylnitrite, pentylnitrite, nitrite salts, ion paired nitrite, silver nitrite, zinc nitrite, iron nitrite, copper nitrite, transition metal-nitrite compounds, or combinations thereof.

In other embodiments, the nitric oxide precursor comprises the reaction product of a thioester, a primary amine, and the nitrosating compound. The thioester may comprise a thiolactone. In certain embodiments, the thiolactone is an amine-containing thiolactone (e.g., thietanone). The primary amine may comprise a cysteine or derivative thereof, such as cysteine, glutathione, acetyl cysteine, penicillamine, acetylpenicillamine, S-nitroso-N-acetylpenicillamine, bucillamine, or combinations thereof.

In still other embodiments, the nitric oxide precursor comprises the reaction product of a primary amine and the nitrosating compound. The primary amine may comprise a cysteine or derivative thereof, such as cysteine, glutathione, acetyl cysteine, penicillamine, acetylpenicillamine, S-nitroso-N-acetylpenicillamine, bucillamine, or combinations thereof.

In these and other embodiments, the epoxy-functional group of the epoxy silane and the primary amine react to form the pendant nitric oxide precursor, while the amino-functional group of the aminosilane and the thioester or thiolactone react to form the pendant nitric oxide precursor, with the precursor being bound to the silane via an amide bond.

Therefore, a method of forming functionalized polymeric material with pendant nitric oxide precursors are also provided herein. The method comprises providing the base polymeric material having a carbonyl-functional group, a siloxy-functional group, or a combination thereof. The method further comprises providing a silane having an amino-functional group or an epoxy-functional group, or combinations thereof. The method further comprises reacting the base polymeric material and the silane to form the pendant amino-functional group, the pendant epoxy-functional group, or combinations thereof, on the base polymeric material.

The inventor contemplates that the step of reacting the base polymeric material and the silane may comprise combining the base polymeric material and the solvent to form a mixture and then combining the mixture and the silane to form the pendant amino-functional group on the base polymeric material. Likewise, the inventor also contemplates that the step of combining the base polymeric material and the solvent may be further defined as combining the base polymeric material, the solvent, and the catalyst to form the pendant amino-functional group, the pendant epoxy-functional group, or combinations thereof, on the base polymeric material.

Consequently, a method of forming a functionalized polymeric material for providing nitric oxide is also contemplated. Such method comprises providing the base polymeric material having a carbonyl-functional group, a siloxy-functional group, or a combination thereof. The method further comprises providing the silane having an amino-functional group, an epoxy-functional group, or combinations thereof. The method further comprises reacting the base polymeric material and the silane to form the pendant amino-functional group, the pendant epoxy-functional group, or combinations thereof, on the base polymeric material. The method further comprises reacting the pendant amino-functional group, the pendant epoxy-functional group, or combinations thereof, and a nitric oxide precursor to form the pendant nitric oxide precursor group on the base polymeric material.

The inventor contemplates that the step of reacting the pendant amino-functional group and the nitric oxide precursor may comprise reacting the pendant amino-functional group and a thioester or thiolactone to form the pendant thiol-functional group on the base polymeric material. Similarly, the inventor contemplates that the step of reacting the pendant epoxy-functional group and the nitric oxide precursor may comprise reacting the pendant epoxy-functional group and a primary amine containing a thiol group to form the pendant thiol-functional group on the base polymeric material. The method may further comprise reacting the pendant thiol-functional group and a nitrosating compound to form the pendant nitric oxide precursor group on the base polymeric material.

### BRIEF DESCRIPTION OF THE DRAWINGS

FIGS. 1A-1E are photographs illustrating non-limiting embodiments of exemplary functionalized polymeric materials in solution of (A) SNAP-PCPDMS, (B) SNAP-PUPDMS, (C) SNAP-PU, (D) SNAP-EVA, and (E) SNAP-pfPDMS.

FIG. 2A is a photograph illustrating a non-limiting embodiment of an exemplary functionalized polymeric material of SNAP-PCPDMS the polymer (b) and NO release.

FIG. 2B is a graph illustrating NO release by a non-limiting embodiment of an exemplary functionalized polymeric material of SNAP-PCPDMS.

FIG. 3A is a photograph illustrating a non-limiting embodiment of an exemplary functionalized polymeric material of SNAP-PUPDMS the polymer (b) and NO release.

FIG. 3B is a graph illustrating NO release by a non-limiting embodiment of an exemplary functionalized polymeric material of SNAP-PUPDMS.

FIG. 4A is a photograph illustrating a non-limiting embodiment of an exemplary functionalized polymeric material of SNAP-PU the polymer (b) and NO release.

FIG. 4B is a graph illustrating NO release by a non-limiting embodiment of an exemplary functionalized polymeric material of SNAP-PU.

FIG. 5A is a photograph illustrating a non-limiting embodiment of an exemplary functionalized polymeric material of SNAP-EVA the polymer (b) and NO release.

FIG. 5B is a graph illustrating NO release by a non-limiting embodiment of an exemplary functionalized polymeric material of SNAP-EVA.

FIG. 6A is a photograph illustrating a non-limiting embodiment of an exemplary functionalized polymeric material of SNAP-pfPDMS the polymer (b) and NO release.

FIG. 6B is a graph illustrating NO release by a non-limiting embodiment of an exemplary functionalized polymeric material of SNAP-pfPDMS.

FIG. 7A is a graph illustrating tensile properties of a comparative base polymeric material of PCPDMS.

FIG. 7B is a graph illustrating tensile properties of a non-limiting embodiment of an exemplary functionalized polymeric material of SNAP-PCPDMS.

### DETAILED DESCRIPTION

There are a number of examples of surface modifications that have been made to cured polymeric materials using silanes. However, these systems exclusively required the treatment of the cured polymeric materials with plasma or other mechanisms to generate transient surface hydroxyl groups. These hydroxyl groups were then exposed to the silane agent to covalently link the silane to the surface of the material. As a consequence, the plasma treatment of the cured polymeric materials to form the hydroxyl groups led to loss of desirable mechanical, chemical, and/or surface properties, including exclusive top layer modification that impart only surface properties. Moreover, as such treatments are limited to surface modification, the concentration and density of hydroxyl groups for attachment of silane groups is limited, and the subsequent exposure of silane groups on the surface to environmental conditions can still further reduce the number of functionally active silane groups. Still further, plasma generated surface hydroxyl groups are short-lived and must be reacted immediately with a silane agent, rendering manufacturing challenging and often ineffective.

After linking of the silane groups to the surface of thusly treated polymeric material, the silane groups may be reacted with nitric oxide precursors to functionalize the polymeric material for nitric oxide release. However, as noted above, since the polymeric materials are already cured prior to plasma treatment, these polymers are limited in their ability to release meaningful (e.g., antimicrobially effective) quantities of nitric oxide due to the presence of the nitric oxide precursors exclusively at the surface of the cured polymeric material.

The present disclosure overcomes all of these disadvantages and drawbacks and now allows for modified polymeric materials that can contain substantial quantities of nitric oxide precursor groups within and on the surface of a variety of polymeric materials. Moreover, the preparation of such modified polymeric materials does not require curing of the precursor materials but allows already manufactured polymers to be modified to so include a large number of nitric oxide precursor groups. Advantageously, such modification will not substantially affect various mechanical and/or physical properties while still providing significant quantities of nitric oxide precursor groups. Indeed, the so generated materials have the capability to release nitric oxide in antimicrobially effective quantities to reduce viable cell count of pathogens in and on the materials over an extended period of time, and in some cases to even effect sterilization of the materials.

Except in the examples, or where otherwise expressly indicated, all numerical quantities in this description indicating amounts of material or conditions of reaction and/or use are to be understood as modified by the word “about” in describing the broadest scope of the disclosure. In various embodiments, the terms “about” and “approximately”, when referring to a specified, measurable value (such as a parameter, an amount, a temporal duration, and the like), is meant to encompass the specified value and variations of and from the specified value, such as variations of +/−10% or less, alternatively +/−5% or less, alternatively +/−1% or less, alternatively +/−0.1% or less of and from the specified value, insofar as such variations are appropriate to perform in the disclosed embodiments. Thus, the value to which the modifier “about” or “approximately” refers is itself also specifically disclosed.

Practice within the numerical limits stated is generally preferred. Also, unless expressly stated to the contrary: percent, “parts of,” and ratio values are by weight; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

It must also be noted that, as used in the specification and the appended claims, the singular form “a,” “an,” and “the” comprise plural referents unless the context clearly indicates otherwise. For example, reference to a component in the singular is intended to comprise a plurality of components.

As used herein, an “embodiment” means that a particular feature, structure, or characteristic is included in at least one or more manifestations, examples, or implementations of this invention. Furthermore, the particular features, structures or characteristics may be combined in any suitable manner, as would be apparent to a person skilled in the art. Combinations of features of different embodiments are all meant to be within the scope of the invention, without the need for explicitly describing every possible permutation by example. Thus, any of the claimed embodiments can be used in any combination.

As used herein, the term “weight percent” (and thus the associated abbreviation “wt. %”) typically refers to a percent by weight expressed in terms of a weight of dry matter. As such, it is to be appreciated that a wt. % can be calculated on a basis of a total weight of a composition or calculated from a ratio between two or more components/parts of a mixture (e.g., a total weight of dry matter).

As used herein, the term “substantially” refers to the complete, or nearly complete, extent or degree of an action, characteristic, property, state, structure, item, or result. As an arbitrary example, an object that is “substantially” enclosed would mean that the object is either completely enclosed or nearly completely enclosed so as to have the same overall result as if the object were completely enclosed.

The drawings are semi-diagrammatic and not to scale and, particularly, some of the dimensions are for the clarity of presentation and are shown exaggerated in the drawings. Similarly, although the views in the drawings for ease of description generally show similar orientations, this depiction in the drawings is arbitrary. Generally, functionalized polymeric materials can be operated in any orientation. As used herein, it will be understood that when a first element or layer is referred to as being “over,” “overlying,” “under,” or “underlying” a second element or layer, the first element or layer may be directly on the second element or layer, or intervening elements or layers may be present where a straight line can be drawn through and between features in overlying relationship. When a first element or layer is referred to as being “on” a second element or layer, the first element or layer is directly on and in contact with the second element or layer. Further, spatially relative terms, such as “upper,” “over,” “lower,” “under,” and the like, may be used herein for ease of description to describe one element or feature's relationship to another element(s) or feature(s) as illustrated in the figures. It will be understood that the spatially relative terms are intended to encompass different orientations of the functionalized polymeric material in use or operation in addition to the orientation depicted in the figures. For example, if the functionalized polymeric material in the figures is turned over, elements described as being “under” other elements or features would then be oriented “above” the other elements or features. Thus, the exemplary term “under” can encompass either an orientation of above or below. The functionalized polymeric material may be otherwise oriented (rotated 90 degrees or at other orientations) and the spatially relative descriptors used herein may likewise be interpreted accordingly.

Throughout this disclosure, where publications are referenced, the disclosures of these publications in their entireties are hereby incorporated by reference into this disclosure to more fully describe the state of the art to which this disclosure pertains.

The following detailed description is merely illustrative in nature and is not intended to limit the embodiments of the subject matter or the application and uses of such embodiments. Furthermore, there is no intention to be bound by any expressed or implied theory presented in the preceding technical field, background, brief summary or the following detailed description.

A functionalized polymeric material capable of forming and releasing nitric oxide is provided herein. The functionalized polymeric material comprises a pendant amino-functional group, a pendant epoxy-functional group, or a combination thereof, that may be used to introduce a pendant nitric oxide precursor group. The pendant nitric oxide precursor group of the functionalized polymeric material is capable of decomposing to form nitric oxide. In various embodiments, the nitric oxide and air will react, resulting in a mixture containing various oxides of nitrogen. Specifically, the addition of nitric oxide to air, or air to nitric oxide, results in the formation of nitric dioxide when nitric oxide reacts with the oxygen in air. The concentration of each nitrogen-oxide species that is present in a mixture may vary with temperature, pressure, and initial concentration of the nitric oxide.

Nitric oxide is lipid soluble and has the ability to disrupt the lipid membranes of microorganisms, to modulate cell and tissue response, to control coagulation and biological integration, to impart antimicrobial characteristics, etc. Furthermore, nitric oxide may inactivate thioproteins thereby disrupting the functional proteins of microbes. Nitrogen dioxide is more water soluble than nitric oxide. Finally, nitric oxide and nitrogen dioxide are effective disruptors of DNA, causing strand breaks and other damage leading to an inability for the cell to function.

As used herein, the term “nitric oxide” or “NO” means the NO free radical. As is well known, NO is chemically instable and readily reacts with oxygen to form various oxides, collectively referred to as NOx. As used herein, the term NOx is an abbreviation for nitrogen oxides or the oxides of nitrogen, which are the oxides formed by nitrogen in which nitrogen exhibits each of its positive oxidation numbers from +1 to +5. As used herein, the terms “nitrogen oxides” and ‘oxides of nitrogen’ and ‘NOx’ mean a gas having one or more of the following gases, all of which contain nitrogen and oxygen in varying amounts: nitric oxide (NO), nitrogen dioxide (NO₂), nitrogen trioxide (NO₃), dinitrogen trioxide (N₂O₃), dinitrogen tetroxide (N₂O₄), dinitrogen pentoxide (N₂O₅) and nitrous oxide (N₂O). As used herein, the phrase “nitric oxide precursor” means a compound or composition capable of producing or releasing NO, NO₂, and NOx.

The functionalized polymeric material may be used in a wide variety of medical and consumer applications. As described in greater detail below, the properties of the functionalized polymeric material may be adjusted to suit specific applications. Non-limiting examples of suitable adjustments include changing chemical and physical configurations of functionalized polymeric material, nitric oxide generation capabilities, the rate of release of nitric oxide, mechanical properties of the functionalized polymeric material, surface chemistry of the functionalized polymeric material, hydrophobicity of the functionalized polymeric material, etc. In particular, the ability to combine nitric oxide release with the base polymeric material to form the functionalized polymeric material capable of providing controlled nitric oxide release is useful for the development of medical and consumer devices. Therefore, polymeric materials suitable for modification as described herein especially include polymers approved by the FDA for use with drugs, medical devices, and food.

Viewed from a different perspective, the inventor contemplates utilizing the functionalized polymeric material to form nitric oxide for a variety of medical and consumer applications. In various embodiments, an object that is cast, molded, or milled, a film, a coating, comprising, or formed from, the functionalized polymeric material may be utilized. Non-limiting examples of suitable uses of the functionalized polymeric material (e.g., as films or coatings) include; hygienic containers for sanitizing hygiene devices (e.g., toothbrushes, mouth/bite guards, CPAP masks, facemasks, and the like); medical device containers for sanitizing medical instruments (e.g., stethoscopes, otoscopes, and the like), medical devices (e.g., portable ultrasound device, communication devices, and the like); components of devices exposed to moisture for resisting growth of mold or mildew (e.g., washing machines, boat compartments, and the like); sporting equipment (e.g., yoga mats, touchable surfaces of strength training equipment, touchable surfaces of cardio equipment, and the like); liners for sporting equipment bags for sanitizing sporting equipment (e.g., shoes, hockey equipment, ski equipment, facemasks, googles, helmets, and the like); food packaging for preserving foodstuff (e.g., meats, fruits, vegetables, cheeses, ingredients thereof, and the like); components of vehicles for sanitizing vehicles (e.g., headliners, seat cushion liners, carpet liners, and the like); and within drawers of cabinets, desks, boxes, etc., to eliminate musty odors.

With respect to the functionalized polymeric material, the inventor contemplates that the functionalized polymeric material comprises, consists essentially of, or consists of, the reaction product of a base polymeric material and a silane. To this end, the base polymeric material and the silane react to form the pendant amino-functional or epoxy-functional group on the base polymeric material. Advantageously, the functionalized polymeric material may exhibit an increased solubility in organic solvent (e.g., ethyl acetate, propyl acetate, hexanes, etc.) as compared to the solubility of the base polymeric material prior to functionalization in the organic solvent as determined in accordance with ASTM 3132-84.

In certain embodiments, the inventor contemplates that the functionalized polymeric material comprises, consists essentially of, or consists of, the reaction product of the base polymeric material, the silane, and a nitric oxide precursor. To this end, the base polymeric material, the silane, and the nitric oxide precursor react to form the pendant nitric oxide precursor group on the base polymeric material. In particular, as introduced above, the base polymeric material and the silane react to form the pendant amino-functional group, the pendant epoxy-functional group, or a combination thereof, on the base polymeric material, and then the pendant amino-functional group, the pendant epoxy-functional group, or a combination thereof, and the nitric oxide precursor react to form the pendant nitric oxide precursor group on the base polymeric material. In various embodiments, the inventor observed that the pendant nitric oxide precursor group exhibits an improved stability as compared to the stability of the nitric oxide precursor separate from the base polymeric material. More specifically, in these and other embodiments, the pendant nitric oxide precursor group exhibits a reduced rate of decomposition to nitric oxide as compared to rate of decomposition of the nitric oxide precursor to nitric oxide.

The base polymeric material has a carbonyl-functional group, a siloxy-functional group, or combinations thereof. To this end, the base polymeric material may comprise a moiety/repeat unit having the carbonyl-functional group or the siloxy-functional group. The moiety may be a urethane moiety, an ester moiety, a carbonate moiety, a siloxane moiety, or combinations thereof. However, it is to be appreciated that any moiety including an oxo group (carbonyl oxygen) may be included in the base polymeric material as contemplated herein.

In various embodiments, the base polymeric material is substantially free of hydroxyl-functional groups. The term “substantially” as utilized in this context means that the base polymeric material has a hydroxyl value of less than 50, less than 40, less than 30, less than 20, less than 10, less than 5, less than 1, or less than 0.1 mg KOH per gram, as determined in accordance with ASTM 4274 In contrast to conventional polymeric materials that utilize plasma or other high-energy mechanisms to generate transient surface hydroxyl groups, the carbonyl-functional group or the siloxy-functional group of the base polymeric material contemplated herein reacts with the pendant amino-functional group, typically in a organometal catalyzed reaction. Without being bound by theory, the inventor believes that this association of the carbonyl-functional group or the siloxy-functional group with the pendant amino-functional group provides improved properties to the functionalized polymeric material.

In further aspects, the inventor contemplates that the base polymeric material need not be fully cured prior to forming at least the pendant amino-functional group or epoxy-functional group. As described in greater detail below, at least the pendant amino-functional or epoxy-functional group may be formed on the base polymeric material in the presence of a solvent. Said differently, the base polymeric material may be reacted with the silane (most preferably an organosilane) in solution to form the pendant amino-functional group or epoxy-functional group. Without being bound by theory, the inventor believes that such in solution formation of the pendant amino-functional group or epoxy-functional group provides improved properties to the functionalized polymeric material. In this context, it should be noted that the organosilane in preferred embodiments will not act as a crosslinker but is used to introduce a pendant reactive group for attachment of the nitric oxide precursor. Furthermore, in embodiments where the pendant amino-functional group or epoxy-functional group is reacted with the nitric oxide precursor to form the pendant nitric oxide precursor group on the base polymeric material, the nitric oxide precursor group is not limited to the surface of the functionalized polymeric material. Therefore, improved control of nitric oxide release can be achieved by adjusting the mechanical, chemical, structural, and surface properties of the functionalized polymeric material.

For example, Scheme 1 below exemplarily depicts introduction of a pendant amine group into a polyurethane polymer using a reaction in which the carbonyl oxygen of the polymer backbone reacts with 3-aminopropyl trimethoxy siloxane.

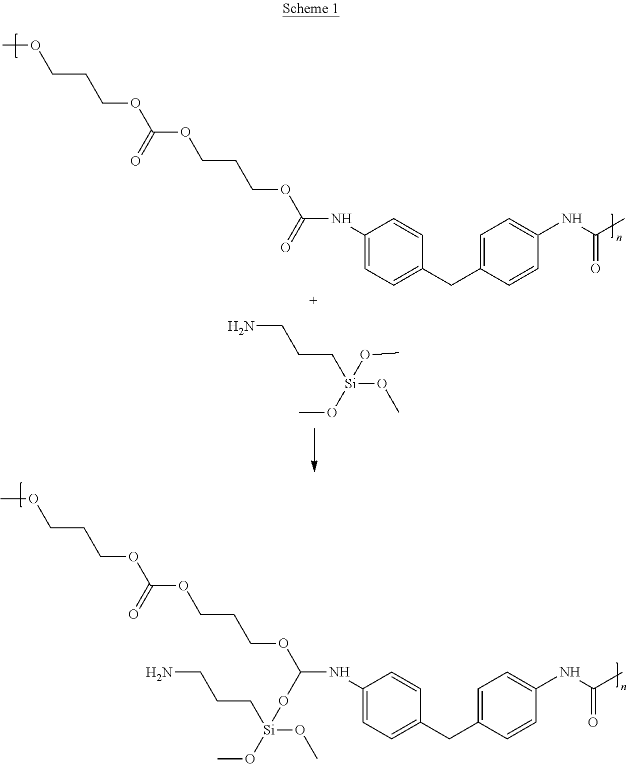

For example, Scheme 2 below exemplarily depicts introduction of a pendant amine group into a polyamide polymer using a reaction in which the carbonyl oxygen of the polymer backbone reacts with 3-aminopropyl trimethoxy siloxane.

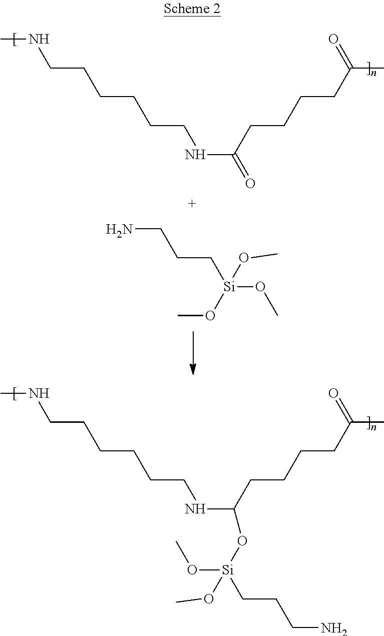

For example, Scheme 3 below exemplarily depicts introduction of a pendant amine group into an EVA polymer using a reaction in which the carbonyl oxygen of the acetate group ester in the repeat unit of the EVA reacts with 3-aminopropyl trimethoxy siloxane.

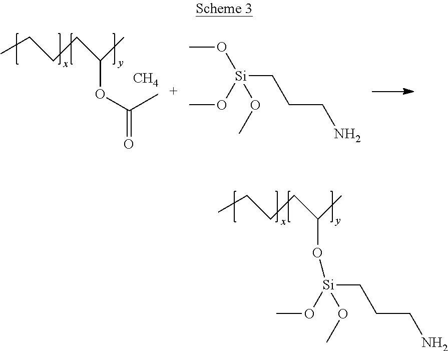

For example, Scheme 4 below exemplarily depicts introduction of a pendant amine group into a PDMS polymer using a reaction in which the hydroxyl oxygen of the polymer backbone reacts with 3-aminopropyl trimethoxy siloxane without increasing crosslinking.

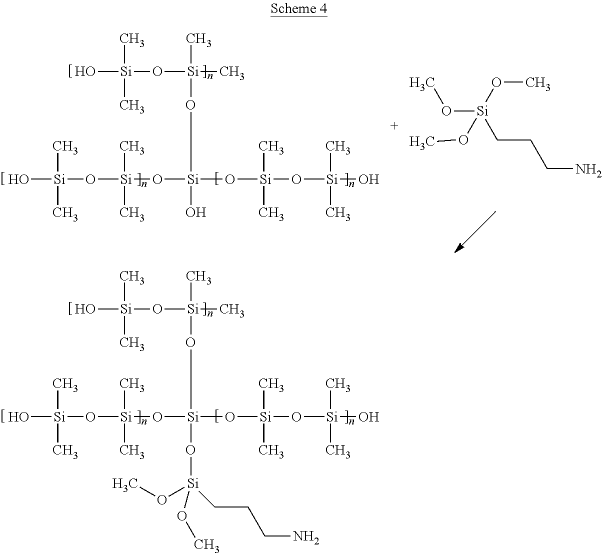

For example, Scheme 5 below exemplarily depicts introduction of a pendant amine group into an epoxy polymer using a reaction in which the carbonyl oxygen of the polymer backbone reacts with 3-aminopropyl trimethoxy siloxane.

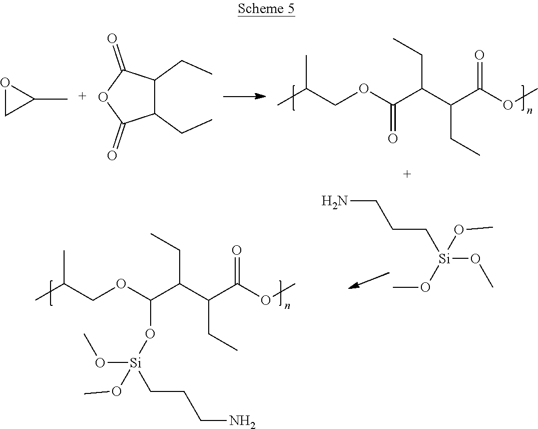

Moreover, the so functionalized polymeric material will be capable of carrying significantly increased quantities of nitric oxide precursor groups as compared to the same polymeric material that is only surface modified with conventional plasma-based modification. Most typically, the materials presented herein will carry at least 10%, or at least 25%, or at least 40%, or at least 60%, or at least 80%, or at least 100%, or at least 150%, or at least 200% more nitric oxide precursor groups per weight unit of a cube-shaped material sample.

In one embodiment, the base polymeric material comprises a carbonate moiety and a siloxane moiety. This base polymer material may be in the form of a copolymer comprising carbonate blocks and siloxane blocks. The carbonate blocks and the siloxane blocks may be present in any amount relative to each other. The copolymer may have a viscosity average molecular weight in the amount of from about 1,000 to about 100,000 or alternatively from about 10,000 to about 50,000. In certain embodiments, the base polymeric material comprises a polycarbonate-polydimethylsiloxane block copolymer. In these and other embodiments, the polydimethylsiloxane utilized to form the polycarbonate-polydimethylsiloxane block copolymer is hydroxy terminated to increase crosslinking within the base polymer material.

In another embodiment, the base polymeric material comprises a urethane moiety and a siloxane moiety. This base polymer material may be in the form of a copolymer comprising urethane blocks and siloxane blocks. The urethane blocks and the siloxane blocks may be present in any amount relative to each other. The copolymer may have a viscosity average molecular weight in the amount of from about 1,000 to about 100,000 or alternatively from about 10,000 to about 50,000. In certain embodiments, the base polymeric material comprises a polyurethane-polydimethylsiloxane block copolymer. In these and other embodiments, the polydimethylsiloxane utilized to form a polyurethane-polydimethylsiloxane block copolymer is hydroxy terminated to increase crosslinking within the base polymer material.

In yet another embodiment, the base polymer material comprises urethane moieties. This base polymer material may be in the form of a homopolymer comprising urethane blocks. The homopolymer may have a viscosity average molecular weight in the amount of from about 1,000 to about 100,000 or alternatively from about 10,000 to about 50,000. In certain embodiments, the base polymeric material comprises a polyurethane.

In still another embodiment, the base polymer material comprises siloxane moieties. This base polymer material may be in the form of a homopolymer comprising siloxane blocks. The homopolymer may have a viscosity average molecular weight in the amount of from about 1,000 to about 100,000 or alternatively from about 10,000 to about 50,000. In certain embodiments, the base polymeric material comprises a polydimethylsiloxane.

In a further embodiment, the base polymer material comprises ester moieties. This base polymer material may be in the form of a copolymer comprising ethylene blocks and vinyl acetate blocks. The ethylene blocks and the vinyl acetate blocks may be present in any amount relative to each other. The copolymer may have a viscosity average molecular weight in the amount of from about 1,000 to about 100,000 or alternatively from about 10,000 to about 50,000. In certain embodiments, the base polymeric material comprises a poly(ethylene-co-vinyl acetate) copolymer (EVA).

It is to be appreciated that depending on the combination and number of functional groups included in the base polymeric material, the rate and quantity of nitric oxide release for the functionalized polymeric material can be adjusted. In particular, blends of different polymeric materials including various functional groups may be utilized to achieve a desired rate and quantity of nitric oxide release. In addition, or alternatively to, controlling the type and number of functional groups of the polymer material, the rate and quantity of nitric oxide release from the functionalized polymeric material may be controlled by varying the degree of cross-linking of the polymer material. Generally, a lesser degree of cross-linking provides a more porous polymeric structure. While this may have no impact on the number of functional groups, it can provide a more rapid and greater degree of nitric oxide release from the functionalized polymer because the pendant nitric oxide precursor groups are more accessible to the environment. Increasing the cross-linking of the base polymeric material may decrease the porosity of the functionalized polymeric material, which serves to inhibit access to the pendant nitric oxide precursor groups. Thus, various rates of nitric oxide release may be obtained by controlling the access to the pendant nitric oxide precursor groups through the degree of cross-linking of the base polymeric material.

Referring back to the (organo)silane utilized to form the functionalized polymeric material, the inventor contemplates that in some embodiments the silane has an amino-functional group (“aminosilane”). Such aminosilanes may include primary aminosilanes, secondary aminosilanes, tertiary aminosilanes, quaternary aminosilanes, multi-podal (e.g., dipodal) aminosilanes, or combinations thereof. The aminosilane may include substantially any suitable aminosilane, for example, a propyl group containing aminosilane, or an aminosilane compound including a propyl amine. Examples of suitable aminosilanes may include bis(2-hydroxyethyl)-3-aminopropyl trialkoxysilane, diethylaminomethyltrialkoxysilane, (N,N-diethyl-3-aminopropyl)trialkoxysilane), 3-(N-styrylmethyl-2-aminoethylaminopropyl trialk-oxysilane, aminopropyl trialkoxysilane, (2-N-benzylaminoethyl)-3-aminopropyl trialkoxysilane), trialkoxysilyl propyl-N,N,N-trimethyl ammonium, N-(trialkoxysilylethyl)benzyl-N,N,N-trimethyl ammonium, (bis(methyldialkoxysilylpropyl)-N-methyl amine, bis(trialkoxysilylpropyl)urea, bis(3-(trialkoxysilyl)propyl)-ethylenediamine, bis(trialkoxysilylpropyl)amine, bis(trialkoxysilylpropyl)amine, 3-aminopropyltrialkoxysilane, N-(2-aminoethyl)-3-aminopropylmethyldialkoxysilane, N-(2-aminoethyl)-3-aminopropyltrialkoxysilane, 3-aminopropylmethyldialkoxysilane, 3-aminopropyl-trialkoxysilane, (N-trialkoxysilylpropyl)polyethyleneimine, trialkoxysilylpropyldiethylene-triamine, N-phenyl-3-aminopropyltrialkoxysilane, N-(vinylbenzyl)-2-aminoethyl-3-amino-propyltrialkoxysilane, 4-aminobutyltrialkoxysilane, or combinations thereof. In various embodiments, the aminosilane comprises an aminoalkoxysilane. In exemplary embodiments, the aminoalkoxysilane comprises 3-aminopropyl trimethoxy silane.

The inventor also contemplates that in some embodiments the (organo)silane has an epoxy-functional group (“epoxy silane”). Non-limiting examples of suitable epoxy silanes include gamma-glycidoxypropyl trimethoxysilane, gamma-glycidoxypropyl triethoxysilane, gamma-glycidoxypropyl methyldimethoxysilane, gamma-glycidoxypropyl methyldiethoxysilane, beta-(3,4-expoxycyclohexyl)-ethyl trimethoxysilane, beta-(3,4-expoxycyclohexyl)-ethyl methyl dimethoxysilane, beta-(3,4-expoxycyclohexyl)-ethyl methyl diethoxysilane, beta-(3,4-epoxycyclohexyl)-ethyl triethoxysilane, or combinations thereof. In various embodiments, the silane comprises a glycidoxyalkoxysilane. In exemplary embodiments, the epoxy silane comprises 3-glycidoxypropyl)trimethoxysilane.

Referring now to the nitric oxide precursor, there is a wide variety of nitric oxide precursors that can be used, depending on the design constraints of the desired application of the functionalized polymeric material. The nitric oxide precursor can include, but is not limited to, SNAP-PDMS and other nitric oxide donating polymers that use different nitric oxide moieties and different polymer base materials. The nitric oxide donors can be covalently linked to the polymer or blended into the polymer to form the nitric oxide precursor. Discrete nitric oxide donors can also be used in solid, liquid or gel forms. Non-limiting examples of this include one or more of S-nitroso-N-acetyl-D-penicillamines (SNAP), nitrite, S-nitrosocysteine, S-nitrosoglutathione, diazeniumdiolate compounds, enzymatic generation of NO from arginine, or organitrites, biological sources such a macrophage generation, etc. Non-limiting examples of suitable S-nitroso-N-acetyl-D-penicillamines and other photosensitive S-nitrosothiols covalently attached to polymers are described in U.S. Pat. No. 9,884,943 B2 and International Publication No. WO 2020/018488 A1, which are incorporated by reference in their entirety. Non-limiting examples of other suitable nitric oxide precursors are described in U.S. Pat. App. Pub. No. 2021/0220523 A1, which is incorporated by reference in its entirety.

Other non-limiting examples of nitric oxide precursors include one or more of gas phase delivery from polymers, acidified nitrite or nitrate, nitric oxide donating molecules such as diazeniumdiolates, nitrosothiols, nitrosyl compounds, or other methods of NO generation such as enzymatic production of nitric oxide, chemical production of nitric oxide from ascorbic acid or metal catalysis, electrochemical production of nitric oxide, photolytic cleavage of bonds to release nitric oxide, direct delivery of nitric oxide gas, etc.

As should be evident from the foregoing, the nitric oxide precursor group will be introduced into the polymer via reaction of the nitric oxide precursor group with the pendant amino or epoxy group (that was previously introduced via the aminosilane or epoxy silane), and the type of pendant group will determine the particular type of nitric oxide precursor group. For example, where the pendant group is an epoxy group, the nitric oxide precursor group will typically be a primary amine compound that further includes a thiol group. On the other hand, where the pendant group is an amino group, the nitric oxide precursor group will typically be a thioester or thiolactone. Regardless of the type of compound, it should be appreciated that upon reaction with the pendant amino or epoxy group, the nitric oxide precursor group will initially provide a pendant thiol group that can then be nitrosylated to form the corresponding S-nitrosothiol, which is the source of nitric oxide upon decomposition of the S-nitrosothiol.

In one exemplary embodiment, the nitric oxide precursor comprises the reaction product of an acetylpenicillamine thiolactone and a pendant amino group and a nitrosating compound. The acetylpenicillamine thiolactone and the nitrosating compound may be reacted at any ratio or under any conditions known in the art. In certain embodiments, these components are reacted at room temperature under agitation. However, it is to be appreciated that the reactions conditions may be adjusted depending on the specific materials utilized or the desired properties.

The nitrosating compound may be any compound serving as a source of forming nitroso groups and will in general be a compound of formula NOX where X is an organic or inorganic anion or a group OR₂ where R₂ is an organic group. Alternatively, gaseous nitric oxide may also be used. X may thus be an organic anion derived from a carboxylic acid, e.g., an alkane carboxylic acid containing 2-7 carbon atoms, nitrosating agents of this type including acetyl nitrite and propionyl nitrite. Where X is an inorganic anion, this may be derived from, for example, a mineral acid, e.g., a halide ion such as chloride or bromide or a sulphate ion, or from a Lewis acid, e.g., a borofluoride ion. Other inorganic anions include hydroxide and sulphonate. Nitrosating compounds of this type thus include nitrosyl chloride, nitrosyl sulphate, nitrosyl borofluoride, nitrous acid and Fremys salt (potassium nitrosyldisulphonate). Where X is a group of formula OR₂ the organic group R₂ may be, for example, a lower alkyl group, such as containing 1-9 carbon atoms, e.g., ethyl, n-propyl, isopropyl, n-butyl, t-butyl or isopentyl.

In certain embodiments, the nitrosating compound comprises a nitrite. The nitrite may comprise sodium nitrite, calcium nitrite, potassium nitrite, tetrabutylammonium nitrite, dicyclohexylammonium nitrite, butylnitrite, isobutylnitrite, t-butylnitrite, amylnitrite, pentylnitrite, nitrite salts, ion paired nitrite, silver nitrite, zinc nitrite, iron nitrite, copper nitrite, transition metal-nitrite compounds, or combinations thereof. Also, nitric oxide gas may be used as a nitrosating agent.

In another exemplary embodiment, the nitric oxide precursor comprises the reaction product of a thioester, a primary amine, and the nitrosating compound. The thioester, the primary amine, and the nitrosating compound may be reacted at any ratio or under any conditions known in the art. In certain embodiments, these components are reacted at room temperature under agitation. However, it is to be appreciated that the reactions conditions may be adjusted depending on the specific materials utilized or the desired properties.

The thioester may comprise an alkylthioester, an aralkylthioester, a cyclic thioester, or combinations thereof. Therefore, non-limiting examples of suitable thioesters include thiolactones, thioacetates, thiobenzoates, or combinations thereof. In certain embodiments, the thioester comprises a thiolactone. The thiolactone may be an amine-containing thiolactone (e.g., thietanone). However, it is to be appreciated that any amine-containing thioester may be utilized to form the nitric oxide precursor.

Suitable primary amines may comprise a cysteine or derivative thereof, such as cysteine, glutathione, acetyl cysteine, penicillamine, acetylpenicillamine, S-nitroso-N-acetylpenicillamine, bucillamine, or combinations thereof. It is to be appreciated that the primary amine may be included as part of a peptide, a polymer, a copolymer, or other macromolecules. In embodiments when the cysteine or derivative thereof is utilized as part of a peptide, the peptide may include any combination of amino acids so long as the peptide includes the cysteine or derivative thereof as at least one of the constituents of the peptide. Non-limiting examples of suitable cysteines or derivatives thereof are described in a journal article titled “S-Nitrosothiol Detection via Amperometric Nitric Oxide Sensor with Surface Modified Hydrogel Layer Containing Immobilized Organoselenium Catalyst” (Langmuir 2006, 22, 25, 10830-10836), which is incorporated by reference in its entirety.

Referring back to formation of the functionalized polymeric material, the base polymeric material, the silane, and optionally the nitric oxide precursor may be reacted at any ratio or under any conditions known in the art. In some embodiments, the base polymeric material and the silane may be reacted at a volume ratio of silane to polymeric material of from about 0.1:1 to about 20:1, alternatively from about 0.5:1 to about 10:1, alternatively from about 1:1 to about 5:1, or alternatively from about 2:1 to 3:1. In these and other embodiments, the base polymeric material and the silane may be reacted at room temperature under agitation. However, it is to be appreciated that the reactions conditions may be adjusted depending on the specific materials utilized or the desired properties.

Viewed from a different perspective, various amounts or concentrations of the base polymeric material and the silane may be utilized in a reaction mixture to form the rection product. The reaction mixture may include the base polymeric material in an amount of from about 0.001 to about 1 grams/milliliter (g/mL), alternatively about 0.005 to about 0.5 g/mL, alternatively about 0.01 to about 0.1 g/mL, or alternatively about 0.02 to about 0.08 g/mL. The reaction mixture may include the silane in an amount of from about 0.001 to about 1 grams/milliliter (g/mL), alternatively about 0.005 to about 0.5 g/mL, alternatively about 0.01 to about 0.1 g/mL, or alternatively about 0.02 to about 0.08 g/mL.

In certain embodiments, the reaction product of the base polymeric material, the silane, and optionally the nitric oxide precursor may be formed in the presence of a catalyst. If utilized, the catalyst may be included in various amounts. The catalyst may comprise any suitable catalyst or mixtures of catalysts known in the art. In certain embodiments, the catalyst may comprise a transition metal catalyst (e.g., an organotin catalyst). In exemplary embodiments, the catalyst comprises a dimethyltin dineodecanoate catalyst.

Other non-limiting examples of suitable catalysts include metal catalysts, amine catalysts, and a combination thereof. Examples of suitable metal catalysts include tin, iron, lead, bismuth, mercury, titanium, hafnium, zirconium, iron(II) chloride, zinc chloride, lead octoate stabilized stannous octoate, tin(II) salts of organic carboxylic acids such as tin(II) acetate, tin(II) octoate, tin(II) ethylhexanoate and tin(II) laurate, and dialkyltin(IV) salts of organic carboxylic acids such as dibutyltin dilaurate, dibutyltin diacetate, dibutyltin maleate and dioctyltin diacetate, and combinations thereof. In certain embodiments, the polymerization catalyst component comprises dimethylethanolamine. Examples of suitable amine catalysts include amidines such as 2,3-dimethyl-3,4,5,6-tetrahydropyrimidine, tertiary amines such as triethylamine, tributylamine, dimethylbenzylamine, N-methylmorpholine, S-ethylmorpholine, N-cyclohexylmorpholine, N,N,N′,N′-tetramethylethylenediamine, N,N,N′,N′-tetramethyl-butanediamine, N,N,N′,N′-tetamethylhexane-1,6-diamine, pentamethyldiethylenetriamine, bis(dimethylaminoethyl) ether, bis(dimethylaminopropyl)urea dimethylpiperazine, 1,2-dimethylimidazole, 1-azabicyclo[3.3.0]octane and typically 1,4-diazabicyclo[2.2.2]octane, and alkanolamine compounds such as triethanolamine, triisopropanolamine, N-methyl-diethanolamine and N-ethyldiethanolamine, dimethylethanolamine, and combinations thereof.

In various embodiments, the reaction product of the base polymeric material, the silane, and optionally the nitric oxide precursor may be formed in the presence of a solvent. If utilized, the solvent may be included in various amounts. In certain embodiments, the solvent may comprise an organic solvent, such as tetrahydrofuran. Other non-limiting examples of suitable solvents include aromatics, aliphatics, ketones, such as methyl ethyl ketone, isobutyl ketone, ethyl amyl ketone, acetone, alcohols, such as methanol, ethanol n-butanol isopropanol esters, such as ethyl acetate, glycols, such as ethylene glycol propylene glycol ethers, such as tetrahydrofuran, ethylene glycol mono butyl ether, or combinations thereof.

The functionalized polymeric material may further include a variety of additives, including, but not limited to, ascorbate, reducing equivalents, oxidizing equivalents, acids, bases, pH buffers, ionophores, enzymes, any agent that will impact the formation and stability of thiols (e.g., including disulfide formation or breaking of disulfide bonds), nitrosothiols (e.g., acids/bases, ion mobility, gas permeability, reaction/buffering of NO gas), plasticizers, surfactants, colorants, fillers, or combinations thereof.

The plasticizer may include a plasticizer that may be used to modify various characteristics including, but not limited to, permeability, modifying hydrophobicity, tensile strength, elongation, and the like. The plasticizer includes, but is not limited to, phthalates, trimellitates, benzoates, adipates, sebacates, maleates, citrates, epoxidized vegetable oils, sulfonamides, organophosphates, glycols/polyethers, polymeric plasticizers and polybutenes, or combinations thereof. However, it is to be appreciated that the plasticizer may include any other plasticizer understood in the art so long as the plasticizer is compatible with the components of the functionalized polymeric material.

The plasticizer may be an ester plasticizer. Examples of suitable ester plasticizers include, but are not limited to, dioctyl phthalate (DOP), n-hexyl-n-decyl phthalate (NHDP), n-octyl-decyl phthalate (NODP), di(isononyl) phthalate (DINP), di(isodecyl)phthalate (DIDP), diundecyl phthalate (DUP), di(isotridecyl)phthalate (DTDP), di-2-ethylhexyl adipate (DOA), di-n-octyl-n-decyl adipate (DNODA), diisononyl adipate (DINA), di-2-ethylhexyl azelate (DOZ), di-2-ethylhexyl sebacate (DOS), trioctyl trimellitate (TOTM), trioctyl phosphate (TOP), tricresyl phosphate (TCP), aliphatic polyester plasticizer, aliphatic polyol plasticizer, or combinations thereof. In certain embodiments, the plasticizer component includes trioctyl trimellitate (TOTM). It is to be appreciated the plasticizer may include any phthalate known in the art so long as it is compatible with the functionalized polymeric material.

The surfactant may include anionic surfactants, non-ionic surfactants, cationic surfactants, Zwitterionic surfactants, or combinations thereof. However, it is to be appreciated that the surfactant may include any other surfactant understood in the art so long as the surfactant is compatible with the components of the functionalized polymeric material.

Examples of suitable anionic surfactants include, but are not limited to, fatty alcohol sulphates, alkylphenol sulphates, fatty alcohol ether sulphates, fatty alcohol ether sulphates, alkylphenol ether sulphates, alkylbenzene sulphonic acid, alkyl ether carboxylic acid and salts thereof, alkyl sulphosuccinates, alkyl sulphosuccinamates, phosphate esters, α-olefin sulphonates, or combinations thereof. Examples of suitable non-ionic surfactants include, but are not limited to, alcohol ethoxylates, alkylphenol ethoxylates, polyethylene oxide/polyethylene oxide block copolymers, polyvinyl alcohol, polyvinyl pyrroliddone, sorbitan fatty acid esters, sorbitan ester ethoxylates, or combinations thereof. Examples of suitable cationic surfactants includes, but are not limited to, alkyl dimethylamines, quaternary ammonium compounds, or combinations thereof. In certain embodiments, the surfactant component includes a nonionic surfactant. The nonionic surfactant may include an acetylene glycol surfactant, 2-ethylhexanol, or a combination thereof.

The filler may include any filler that may be used for various objectives including, but not limited to, cost control, rheology control, lubricity modification, as well as to prevent seizing or galling. The filler component may include an inorganic filler. Examples of suitable inorganic fillers include, but are not limited to, powdered nickel, copper, zinc, and aluminum. Suitable mineral fillers include, but are not limited to, talc, calcium carbonate, silicates such as mica, wollastonite, titanium dioxide, quarts, fumed silica precipitated silica, graphite, boron nitride, or combinations thereof. Also included are modifiers such as stearates including zinc stearate, magnesium stearate, sodium stearate, etc.

Other components that may be present in the functionalized polymeric material include minor amounts of antioxidants, inhibitors, defoamers, dispersing aids, heat stabilizers, UV stabilizers, and the like, such as one or more components described in U.S. Patent App. Pub. No. 2004/0258922 A1, in U.S. Pat. No. 9,404,015 B2, and U.S. Pat. No. 10,214,668 B2, the disclosures of which are incorporated herein by reference in their entirety. In various embodiments, one or more of such additives are individually present in the functionalized polymeric material in an amount less than about 5 wt. % based on a total weight of the functionalized polymeric material.

The functionalized polymeric material can be formed utilizing conventional techniques understood in the art. In an exemplary method of forming comprises providing the base polymeric material having the carbonyl-functional group, the siloxy-functional group, or the combination thereof. The method further comprises providing the silane having an amino- or epoxy-functional group. The method further comprises reacting the base polymeric material and the silane to form the pendant amino- or epoxy-functional group on the base polymeric material.

The inventor contemplates that the step of reacting the base polymeric material and the silane may comprise combining the base polymeric material and the solvent to form a mixture and then combining the mixture and the silane to form the pendant amino-functional group, the pendant epoxy-functional group, or a combination thereof, on the base polymeric material. Likewise, the inventor also contemplates that the step of combining the base polymeric material and the solvent may be further defined as combining the base polymeric material, the solvent, and the catalyst to form the pendant amino-functional group, the pendant epoxy-functional group, or a combination thereof, on the base polymeric material.

Another exemplary method of forming comprises providing the base polymeric material having the carbonyl-functional group, the siloxy-functional group, or the combination thereof. The method further comprises providing the silane having an amino-functional group, an epoxy-functional group, or a combination thereof. The method further comprises reacting the base polymeric material and the silane to form the pendant amino-functional group, the pendant epoxy-functional group, or a combination thereof, on the base polymeric material. The method further comprises reacting the pendant amino-functional group, the pendant epoxy-functional group, or a combination thereof, and the nitric oxide precursor to form the pendant nitric oxide precursor group on the base polymeric material.

The inventor contemplates that the step of reacting the pendant amino- or epoxy-functional group and the nitric oxide precursor may comprise reacting the pendant amino- or epoxy-functional group and the acetylpenicillamine thiolactone or primary amine compound with a thiol group (e.g., cysteine) to form the pendant thiol-functional group on the base polymeric material. The step of reacting the pendant amino- or epoxy-functional group and the nitric oxide precursor may further comprise reacting the pendant thiol-functional group and the nitrosating compound to form the pendant nitric oxide precursor group on the base polymeric material.

A film comprising the functionalized polymeric material may be formed by various methods understood in the art. For example, the film can be extruded, cast, laminated, etc.

Likewise, a coating comprising the functionalized polymeric material may be formed by various methods understood in the art. For example, the coating can be sprayed onto an article, the article may be submerged into the coating, or the coating can be applied to the article using a mechanical method (e.g., a brush). Other bulk forms of the functionalized polymeric material may be obtained by evaporating the solvent and then shaping the materials into a desired geometry. It is to be appreciated any other method known in the art for forming a film or coating comprising the functionalized polymeric material may be utilized so long as the methods are compatible with the components of the functionalized polymeric material.

### EXAMPLES

The following examples are included to demonstrate various embodiments as contemplated herein. It should be appreciated by those of skill in the art that the techniques disclosed in the examples which follow represent techniques discovered by the inventor(s) to function well in the practice of the invention, and thus can be considered to constitute desirable modes for its practice. However, those of skill in the art should, in light of the present disclosure, appreciate that many changes can be made in the specific embodiments which are disclosed and still obtain a like or similar result without departing from the spirit and scope of the invention. All percentages are in wt. % and all measurements are conducted at 23° C. unless indicated otherwise.

### Example 1: Exemplary SNAP-PCPDMS

Polycarbonate-polydimethylsiloxane block copolymer was dissolved in THE and reacted with 3-aminopropyl trimethoxy siloxane and a small amount of organotin catalyst. After the reaction mixture became slightly cloudy, an acetylpenicillamine thiolactone was reacted with the pendant amine group. The resultant polymer was then reacted with the organ nitrite t-butyl nitrite to convert the exposed sulfur group to the corresponding S-nitrosothiol.

The resultant green polymer can be cast into films or coated onto devices to deposit the NO releasing functionalized polymeric material.

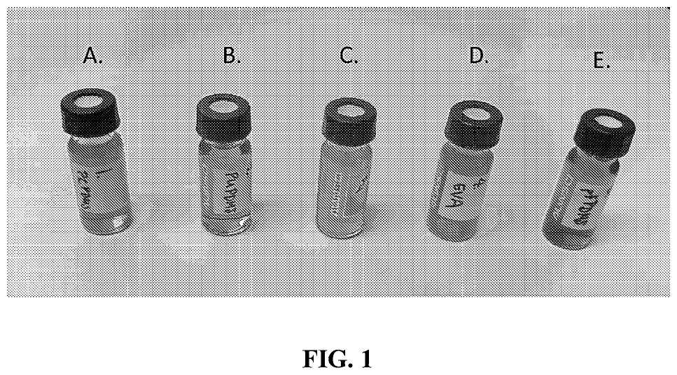

Specific materials: 0.05 g/mL CarboSil20 80 A in THE was reacted with 125 μL 3-aminopropyltrimethoxy silane/mL and 25 μL of dimethyltin dineodecanoate catalyst and stirred for 10 min at room temperature. 10 mg/mL acetylpenicillamine thiolactone were then added and stirred for an additional 10 min. 200 μL tert-butylnitrite were then added/mL of polymer solution. The solution turns a dark green-red color after approximately 5 min. (see FIG. 1A).

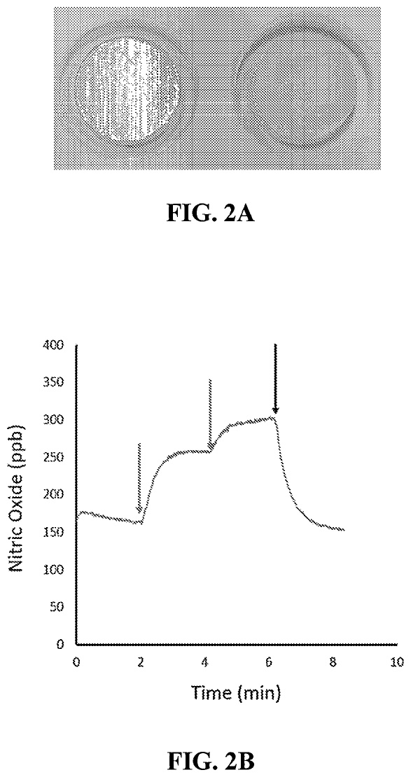

Light initiated NO release was measured form cured polymers films, as illustrated in FIGS. 2A and 2B.

Comparative films formed from base polycarbonate-polydimethylsiloxane block copolymer (“Ctrl 1-4”) and exemplary films formed from nitric oxide precursor functionalized polycarbonate-polydimethylsiloxane block copolymer (“S 1-4”) were evaluated for tensile properties. The tensile properties were evaluated in accordance with ASTM D882-02 using an ElectroForce 3200 mechanical tester.

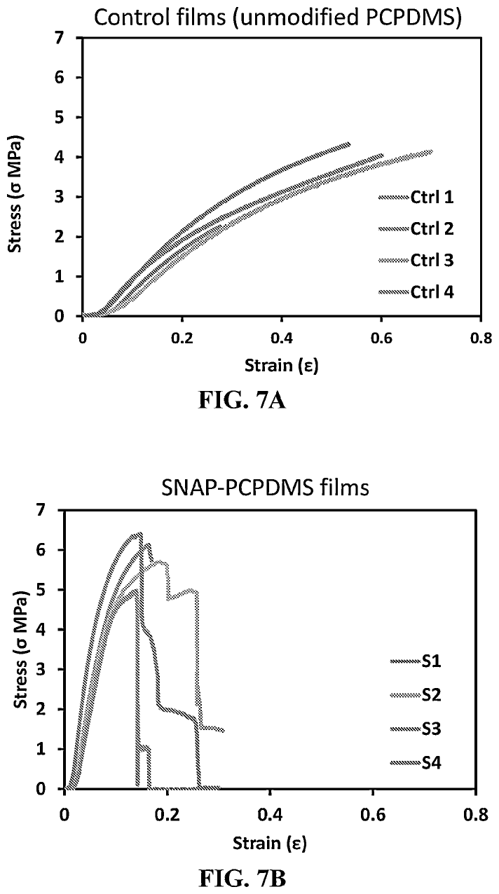

As shown in FIGS. 7A and 7B, the comparative and exemplary films exhibit different tensile properties as exhibited by their stress-strain curves. In general, both films were ductile and suitable for a variety of uses.

### Example 2: Exemplary SNAP-PUPDMS

Polyurethane-polydimethylsiloxane block copolymer was dissolved in THE and reacted with 3-aminopropyl trimethoxy siloxane and a small amount of organotin catalyst. After the reaction mixture became slightly cloudy, an acetylpenicillamine thiolactone was reacted with the pendant amine group. The resultant polymer was then reacted with the organ nitrite t-butyl nitrite to convert the exposed sulfur group to the corresponding S-nitrosothiol.

The resultant green polymer can be cast into films or coated onto devices to deposit the NO releasing functionalized polymeric material.

Specific materials: 0.05 g/mL PurSil20 80 A in THE was reacted with 125 μL 3-aminopropyltrimethoxy silane/mL and 25 μL of dimethyltin dineodecanoate catalyst and stirred for 10 min at room temperature. 10 mg/mL acetylpenicillamine thiolactone were then added and stirred for an additional 10 min. 200 μL tert-butylnitrite were then added/mL of polymer solution. The solution turns a dark green-red color after approximately 5 min. (see FIG. 1B).

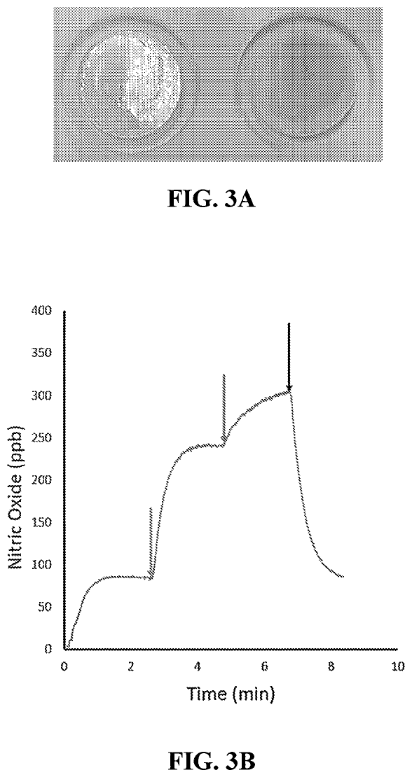

Light initiated NO release was measured form cured polymers films, as illustrated in FIGS. 3A and 3B.

### Example 3: Exemplary SNAP-PU

Polyurethane was dissolved in THE and reacted with 3-aminopropyl trimethoxy siloxane and a small amount of organotin catalyst. After the reaction mixture became slightly cloudy, an acetylpenicillamine thiolactone was reacted with the pendant amine group. The resultant polymer was then reacted with the organ nitrite t-butyl nitrite to convert the exposed sulfur group to the corresponding S-nitrosothiol.

The resultant green polymer can be cast into films or coated onto devices to deposit the NO releasing functionalized polymeric material.

Specific materials: 0.05 g/mL Tecoflex SG 80 A in THE was reacted with 125 μL 3-aminopropyltrimethoxy silane/mL and 25 μL of dimethyltin dineodecanoate catalyst and stirred for 10 min at room temperature. 10 mg/mL acetylpenicillamine thiolactone were then added and stirred for an additional 10 min. 200 μL tert-butylnitrite were then added/mL of polymer solution. The solution turns a dark green-red color after approximately 5 min. (see FIG. 1C).

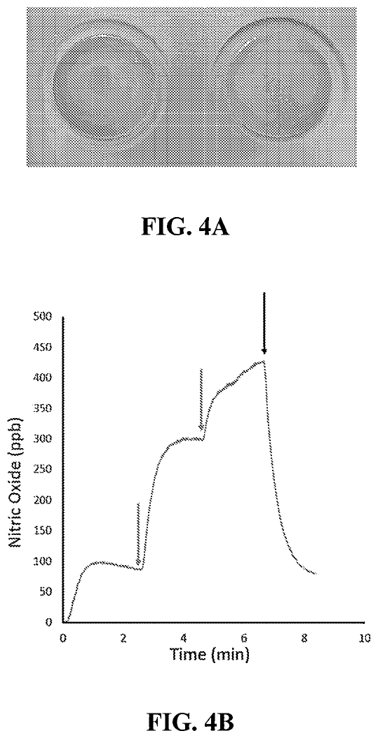

Light initiated NO release was measured form cured polymers films, as illustrated in FIGS. 4A and 4B.

### Example 4: Exemplary SNAP-EVA

Poly(ethylene-co-vinyl acetate) copolymer (EVA) was dissolved in THE and reacted with 3-aminopropyl trimethoxy siloxane and a small amount of organotin catalyst. After the reaction mixture became slightly cloudy, an acetylpenicillamine thiolactone was reacted with the pendant amine group. The resultant polymer was then reacted with the organ nitrite t-butyl nitrite to convert the exposed sulfur group to the corresponding S-nitrosothiol.

The resultant green polymer can be cast into films or coated onto devices to deposit the NO releasing functionalized polymeric material.

Specific materials: 0.05 g/mL EVA (40% vinyl acetate) in THE was reacted with 125 μL 3-aminopropyltrimethoxy silane/mL and 25 μL of dimethyltin dineodecanoate catalyst and stirred for 10 min at room temperature. 10 mg/mL acetylpenicillamine thiolactone were then added and stirred for an additional 10 min. 200 μL tert-butylnitrite were then added/mL of polymer solution. The solution turns a dark green-red color after approximately 5 min. (see FIG. 1D).

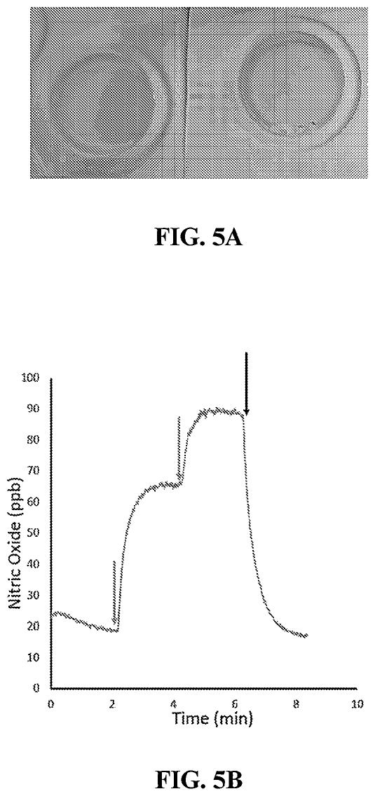

Light initiated NO release was measured form cured polymers films, as illustrated in FIGS. 5A and 5B.

### Example 5: Exemplary SNAP-pfPDMS

Polydimethylsiloxane was dissolved in THE and reacted with 3-aminopropyl trimethoxy siloxane and a small amount of organotin catalyst. After the reaction mixture became slightly cloudy, an acetylpenicillamine thiolactone was reacted with the pendant amine group. The resultant polymer was then reacted with the organ nitrite t-butyl nitrite to convert the exposed sulfur group to the corresponding S-nitrosothiol.

The resultant green polymer can be cast into films or coated onto devices to deposit the NO releasing functionalized polymeric material.

Specific materials: 0.05 g/mL Dow Corning RTV-3140 in THF was reacted with 125 μL 3-aminopropyltrimethoxy silane/mL and 25 μL of dimethyltin dineodecanoate catalyst and stirred for 10 min at room temperature. 10 mg/mL acetylpenicillamine thiolactone were then added and stirred for an additional 10 min. 200 μL tert-butylnitrite were then added/mL of polymer solution. The solution turns a dark green-red color after approximately 5 min. (see FIG. 1E).

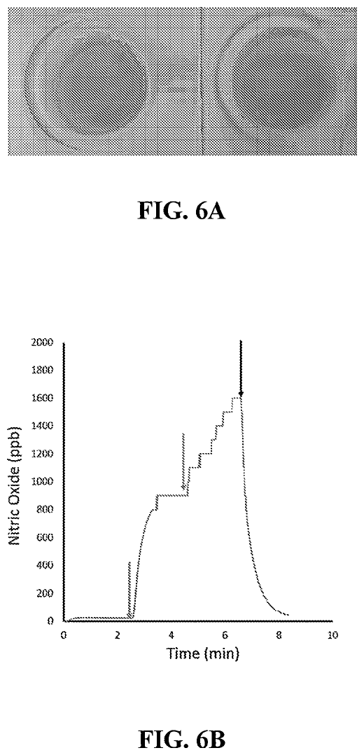

Light initiated NO release was measured form cured polymers films, as illustrated in FIGS. 6A and 6B.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, different, special, and/or unexpected results may be obtained from each member of the respective Markush group independent from all other Markush members. Each member of a Markush group may be relied upon individually and or in combination and provides adequate support for specific embodiments within the scope of the appended claims.

Further, any ranges and subranges relied upon in describing various embodiments of the present invention independently and collectively fall within the scope of the appended claims and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on. As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

1. A method of modifying a polymer, comprising:
providing a polymer comprising a plurality of repeat units, wherein at least some of the repeat units include a carbonyl group or a siloxyl group;
wherein the carbonyl group is part of an amide group (—C(O)NH—) or a carboxylic acid ester group (—C(O)O—) or a carbonate ester group (—O—C(O)O—);
reacting the polymer in the presence of an organometallic catalyst with an organosilane to so produce a silane-modified polymer that includes a pendant amino or epoxy group;
reacting the pendant amino or epoxy group with a sulfur-containing reagent to so produce a thiol-modified polymer that includes a pendant thiol group; and
reacting the pendant thiol group with a nitrosating agent to so produce a S-nitrosothiol containing polymer.

2. The method of claim 1, wherein the carbonyl group or siloxyl group is in a backbone of the polymer.

3. (canceled)

4. The method of claim 1, wherein the carbonyl group is in a side chain of the repeat units.

5. The method of claim 4, wherein the carbonyl group in the side chain is a carboxylic acid ester group (—C(O)O—).

6. The method of claim 1, wherein the polymer is substantially free of hydroxyl groups.

7. The method of claim 1, wherein the polymer is a homopolymer selected from the group consisting of a polyurethane, a polyamide, a polyester, a polycarbonate, and a polysiloxane, or a heteropolymer comprising polymer blocks selected from the group consisting of a polyurethane, a polyamide, a polyester, a polycarbonate, and a polysiloxane.

8. (canceled)

9. The method of claim 1, wherein a first portion of the repeat units include the carbonyl group and wherein a second portion of the repeat units include the siloxyl group, and optionally wherein the polymer is a polycarbonate-polydimethylsiloxane block copolymer or a polyurethane-polydimethylsiloxane block copolymer.

10. (canceled)

11. The method of claim 1, wherein the polymer is a polyurethane, a poly(ethylene-co-vinyl acetate) copolymer, or a polydimethylsiloxane.

12. The method of claim 1, wherein the organosilane is an aminoalkoxysilane or a glycidoxyalkoxysilane, and optionally 3-aminopropyl trimethoxy silane or (3-glycidoxypropyl)-tri-methoxysilane.

13-17. (canceled)

18. The method of claim 1, wherein the amino group is reacted with the sulfur-containing reagent, and wherein the sulfur-containing reagent is a thiolactone or a thioester, or wherein the epoxy group is reacted with the sulfur-containing reagent, and wherein the sulfur-containing reagent is a cysteine, a glutathione, or an acetyl cysteine.

19-27. (canceled)

28. A polymer, comprising:
a polymer comprising a plurality of repeat units, each repeat unit comprising a carbonyl group or a siloxyl group;
wherein the carbonyl group is part of an amide group (—C(O)NH—) or a carboxylic acid ester group (—C(O)O—) or a carbonate ester group (—O—C(O)O—);
wherein at least some of the carbonyl group or a siloxyl groups are modified at respective oxygen atoms to covalently bind an organosilane having a pendant amino or epoxy group; and
wherein at least some of the pendant amino or epoxy groups are modified to covalently bind an organosilane having a pendant amino or epoxy group, or a moiety comprising a S-nitrosothiol group or a thiol group.

29. (canceled)

30. (canceled)

31. The polymer of claim 28, wherein the carbonyl group or siloxyl group is in a backbone of the polymer.

32. (canceled)

33. The polymer of claim 28, wherein the carbonyl group is in a side chain of the repeat units.

34. (canceled)

35. The polymer of claim 28, wherein the polymer is substantially free of hydroxyl groups.

36. The polymer of claim 28, wherein the polymer is a homopolymer selected from the group consisting of a polyurethane, a polyamide, a polyester, a polycarbonate, and a polysiloxane or a heteropolymer comprising polymer blocks selected from the group consisting of a polyurethane, a polyamide, a polyester, a polycarbonate, and a polysiloxane.

37. (canceled)

38. (canceled)

39. The polymer of claim 28 wherein the polymer is a polycarbonate-polydimethylsiloxane block copolymer or a polyurethane-polydimethylsiloxane block copolymer.

40. The polymer of claim 28, wherein the polymer is a polyurethane, a poly(ethylene-co-vinyl acetate) copolymer, or a polydimethylsiloxane.

41-49. (canceled)

50. A method of delivering nitric oxide to a surface of an article, comprising coating or covering the article with a polymer according to claim 28.

51. The method of claim 50, wherein the nitric oxide is delivered to the surface in an amount sufficient to reduce viable microbial count on the surface of the article.

52. The method of claim 50, wherein the nitric oxide is delivered to the surface in an amount sufficient to sterilize the surface of the article.
